(19) United States
(12) Patent Application Publication
(10) Pub. No.: US 2006/0085077 A1
(43) Pub. Date: Apr. 20, 2006

(54) Intervertebral implant and associated method

(75) Inventors: Stephen D. Cook, Baton Rouge, LA (US); Kirk J. Bailey, Blairstown, NJ (US); Gretchen Dougherty Shah, Wayne, NJ (US)

Correspondence Address: HARNESS, DICKEY & PIERCE, P.L.C., P.O. BOX 828, BLOOMFIELD HILLS, MI, 48303, US

(73) Assignee: EBI, L.P.

(21) Appl. No.: 11/248,101

(22) Filed: Oct. 12, 2005

Related U.S. Application Data
(60) Provisional application No. 60/619,842, filed on Oct. 18, 2004.

(51) Int. Cl. A61F 2/44; A61B 17/90; A61F 2/46

(52) U.S. Cl. 623/17.15; 606/90

(57) ABSTRACT

An intervertebral implant and associated method. The intervertebral implant can include a first component having a first articulating surface and a first bone engagement surface for engaging a first vertebra, and a second component having a second articulating surface and a second bone engagement surface for engaging a second vertebra adjacent to the first vertebra. The first and second articulating surfaces articulate with each other for substantially replicating a natural spinal movement including torsion, extension/flexion, and lateral bending. The first and second bone engagement surfaces define an outer surface substantially shaped as an envelope of two intersecting cylinders.

### CROSS-REFERENCE TO RELATED APPLICATIONS

This application claims the benefit of U.S. Provisional Application No. 60/619,842, filed on Oct. 18, 2004. The disclosure of the above application is incorporated herein by reference.

### INTRODUCTION

The spinal column provides the main support for the body and is made of thirty three individual bones called vertebrae. There are twenty four moveable vertebrae in the spine, with the remaining being fused. Each vertebra includes an anterior vertebral body, a posterior vertebral arch that protects the spinal cord, and posterior processes extending from the vertebral arch. The vertebral body is drum-shaped and includes superior and inferior endplates. The moveable vertebrae are stacked in series and are separated and cushioned by anterior intervertebral discs.

Each vertebral body transmits loads to adjacent bodies via an anterior intervertebral disc and two posterior facets. The intervertebral disc is composed of an outer fibrous ring called the annulus. Nucleus pulposus is a gel-like substance housed centrally within the annulus and sandwiched between the endplates of the adjacent vertebral bodies. The annulus operates as a pressure vessel retaining an incompressible fluid. In a healthy disc, the nucleus pulposus acts as hard sphere seated within the nuclear recess (fossa) of the vertebral endplates. This sphere operates the fulcrum (nuclear fulcrum) for mobility in the spine. Stability is achieved by balancing loads in the annulus and the facet joints.

Degenerative disc disease affects the physiology of the disc and may be caused by aging, protrusion of the nucleus into the annulus or endplates, trauma or other causes. The result in either case may produce a reduction of disc height, which in turn, alters the loading pattern in the facets causing symptomatic degeneration of the facet joints, thus reducing stability, and compressing nerves branching out of the spinal column.

Examples of surgical treatments of degenerative disc disease include spinal arthroplasty with total disc replacement that requires a full discectomy or with nucleus replacement that disrupts the annulus. Although these devices can be effective for their intended purposes, it is still desirable to have implants and associated methods that are less disruptive and provide the required degree of stability and mobility to the affected region of the spine.

### SUMMARY

The present teachings provide an intervertebral implant and associated method. The intervertebral implant comprises superior and inferior components mutually articulating to replicate natural spine movement.

In one aspect, the present teachings provide an intervertebral implant that can include a first component having a first articulating surface and a first bone engagement surface for engaging a first vertebra, and a second component having a second articulating surface and a second bone engagement surface for engaging a second vertebra adjacent to the first vertebra. The first and second articulating surfaces can articulate with each other for substantially replicating a natural spinal movement including torsion, extension/flexion, and lateral bending. The first and second bone engagement surfaces can define an outer surface substantially shaped as an envelope of two intersecting cylinders.

The present teaching provide a surgical kit that includes an insertion cannula defining a longitudinal bore, an intervertebral implant pre-loaded within the longitudinal bore, and a retainer for temporarily retaining the intervertebral implant within the longitudinal bore.

The present teachings also provide a method for inserting an intervertebral implant in a disc space. The method includes providing an insertion cannula having a longitudinal bore, preloading the intervertebral implant within the longitudinal bore of the insertion cannula in a substantially fixed position, supporting the insertion cannula relative to the disc space, releasing the intervertebral implant from the substantially fixed position, and implanting the intervertebral implant into the disc space.

Further areas of applicability of the present invention will become apparent from the description provided hereinafter. It should be understood that the description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a sagittal sectional view of an intervertebral implant according to the present teachings, shown implanted in a spine;

FIG. 1A is a coronal end view of an intervertebral implant according to the present teachings, shown implanted in a spine;

FIG. 2 is a coronal end view of a toroidal intervertebral implant according to the present teachings;

FIG. 3 is an isometric view of the intervertebral implant of FIG. 2;

FIG. 4 is a coronal end view of a spherical intervertebral implant according to the present teachings;

FIG. 5 is an isometric view of the intervertebral implant of FIG. 4;

FIG. 6 is a coronal end view of an intervertebral implant according to the present teachings;

FIG. 7 is an isometric view of the intervertebral implant of FIG. 6;

FIG. 8 is a side view of a probe shown in use for locating a nuclear recess;

FIGS. 9A, 9B and 9C illustrate exemplary articulation motions including torsion, extension/flexion, and lateral bending, respectively, for a toroidal intervertebral implant according to the present teachings;

FIG. 10 is an isometric view of an intervertebral implant according to the present teachings, shown implanted;

FIG. 11 is a sagittal sectional view of the intervertebral implant of FIG. 10;

FIG. 12A is an isometric view of a superior component of a toroidal intervertebral implant according to the present teachings;

FIG. 12B is a coronal sectional view of the superior component of the toroidal intervertebral implant of FIG. 12A;

FIG. 12C is an axial view of the superior component of the toroidal intervertebral implant of FIG. 12A;

FIG. 12D is a sagittal sectional view of the superior component of the toroidal intervertebral implant of FIG. 12A;

FIG. 13A is an isometric view of an inferior component of a toroidal intervertebral implant according to the present teachings;

FIG. 13B is a coronal sectional view of the inferior component of the toroidal intervertebral implant of FIG. 13A;

FIG. 13C is an axial view of the inferior component of the toroidal intervertebral implant of FIG. 12A;

FIG. 13D is a sagittal sectional view of the inferior component of the toroidal intervertebral implant of FIG. 13A;

FIG. 14 is a conceptual illustration of constructing a toroidal intervertebral implant according to the present teachings;

FIGS. 15A, 15B and 15C illustrate exemplary articulation motions for a spherical intervertebral implant according to the present teachings

FIG. 16A is a sagittal sectional view of a spherical intervertebral implant according to the present teachings;

FIG. 16B is a coronal sectional view of the spherical intervertebral implant of FIG. 16A;

FIG. 17A is an isometric view of an intervertebral implant according to the present teachings;

FIG. 17B is a front view of an intervertebral implant according to the present teachings;

FIG. 17C is side view of an intervertebral implant according to the present teachings;

FIGS. 18-30 illustrate a method of implanting an intervertebral implant according to the present teachings;

FIG. 31 is a side view of a clip holding an intervertebral implant in a insertion cannula according to the present teachings;

FIG. 32 is a view of a clip holding an intervertebral implant in a insertion cannula according to the present teachings;

FIG. 33 is a plan view of a distraction pin guide according to the present teachings; and

FIG. 34 is a sectional view of a drill guide cannula according to the present teachings.

### DESCRIPTION OF VARIOUS ASPECTS

The following description is merely exemplary in nature and is in no way intended to limit the invention, its application, or uses. For example, although the present teachings are illustrated for intervertebral disc implants, the present teachings can be used for other spine implants, such as intervertebral spacers, for example.

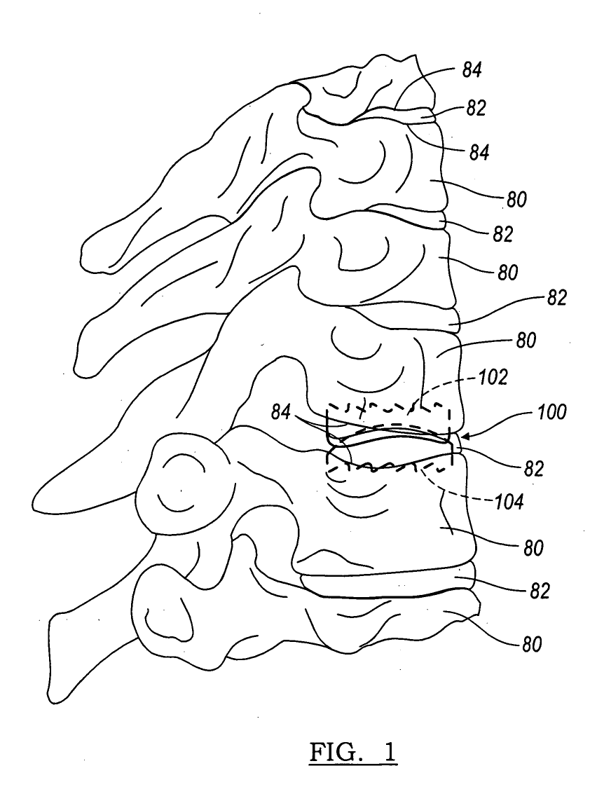

Referring to FIGS. 1 and 1A, exemplary intervertebral implant 100 according to the present teachings are illustrated as implanted between two adjacent vertebral bodies 80 having endplates 84. The intervertebral implant 100 can be nested between the endplates 84 of the vertebral bodies 80 and may be partially surrounded by a portion of a natural intervertebral disc 82 replacing the nucleus thereof. Alternatively, the entire natural intervertebral disc 82 can be removed and replaced by the intervertebral implant 100.

The intervertebral implant 100 can be a multiple component implant that includes superior and inferior components 102, 104 configured for mutual articulation that can replicate the primary modes of motion in the spine and any combination thereof. The superior and inferior articulation components 102, 104 can be designed to resurface the adjacent endplates 84 at the nuclear fulcrum and re-establish disc height to its original dimension. Accordingly, improved motion and increased stability can be established in the region of the intervertebral implant 100 without dependence on the integrity of the endplate cartilage.

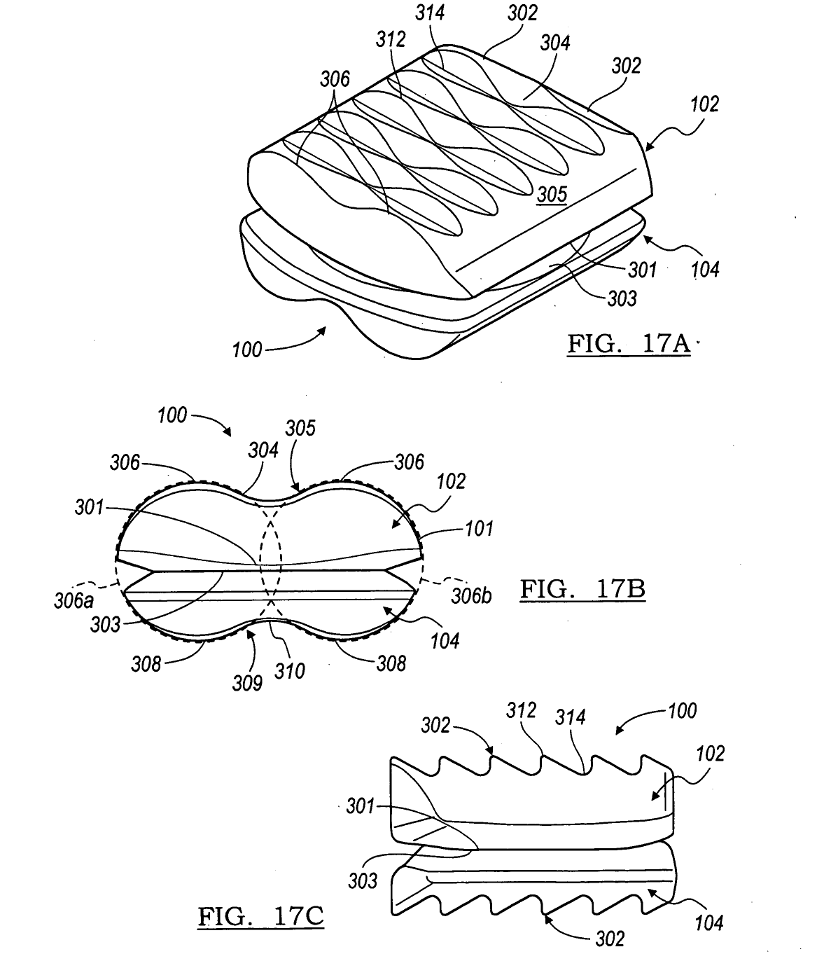

The articulation between the inferior and superior articulation components 102, 104 of the intervertebral implant 100 can substantially replicate natural spinal movement. Two exemplary aspects of such articulation between the inferior and superior articulation components 102, 104 of the intervertebral implant 100 are illustrated in FIGS. 3 and 5, and referred respectively herein as “toroidal” and “spherical” intervertebral implant 100 for reasons that are discussed below. The articulation illustrated in FIG. 1A, and FIGS. 17A-17C is of the spherical type, although toroidal type articulation can also be used with the intervertebral implant 100 illustrated in these figures.

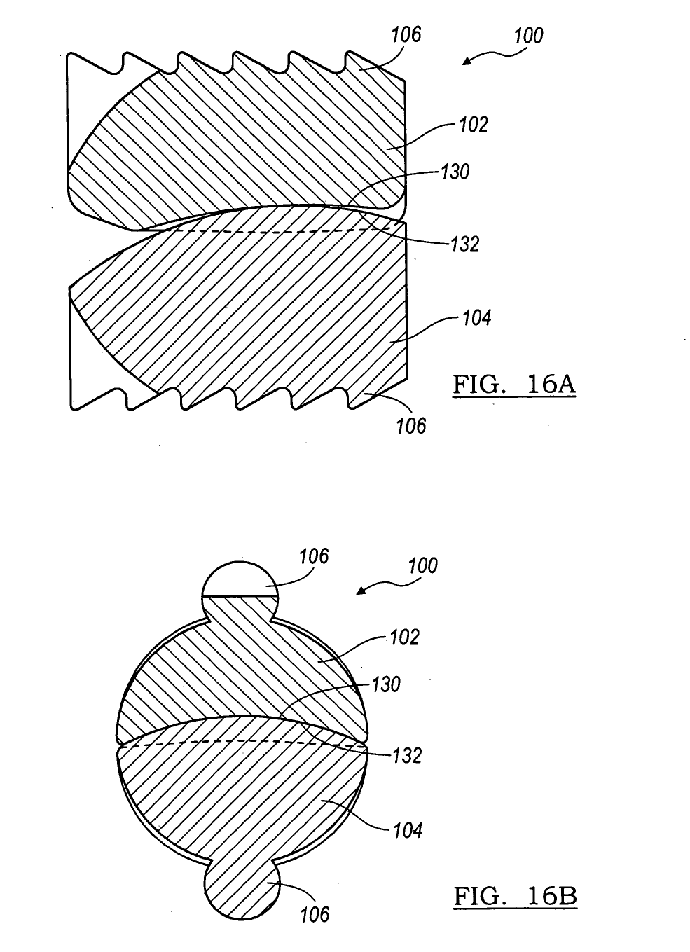

More particularly, FIGS. 9A, 9B, and 9C illustrate respectively torsion, extension/flexion, and lateral bending for the toroidal intervertebral implant 100 of FIG. 3, and FIGS. 15A, 15B, and 15C illustrate respectively torsion, extension/flexion, and lateral bending for the spherical intervertebral implant 100 of FIG. 5.

Referring to FIGS. 3 and 5, each of the superior and inferior components 102, 104 can include a serrated rack 106 for preventing migration of the intervertebral implant 100 relative to the vertebral bodies 80. It will be appreciated that other anchoring structures known in the art can be used for securing the intervertebral implant 100 against migration, such as, for example, projections of various geometric shapes engaging corresponding recesses in the endplates, surface treatment promoting frictional resistance including porous coatings that promote bone growth, and other structures.

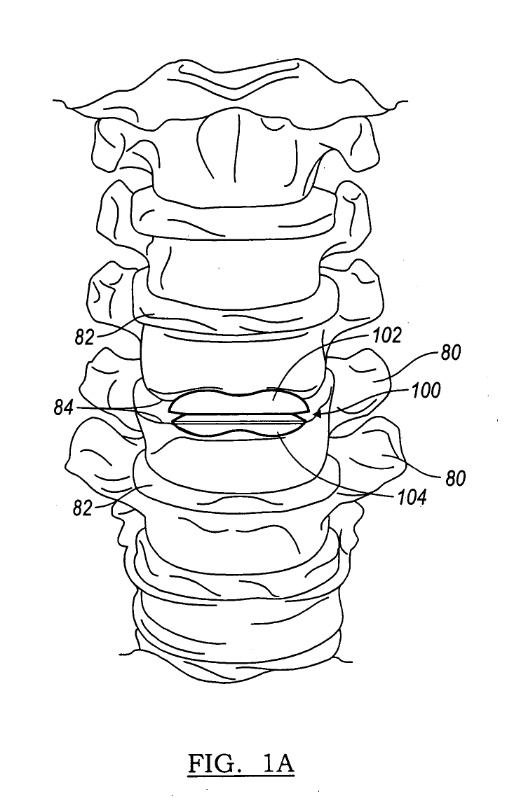

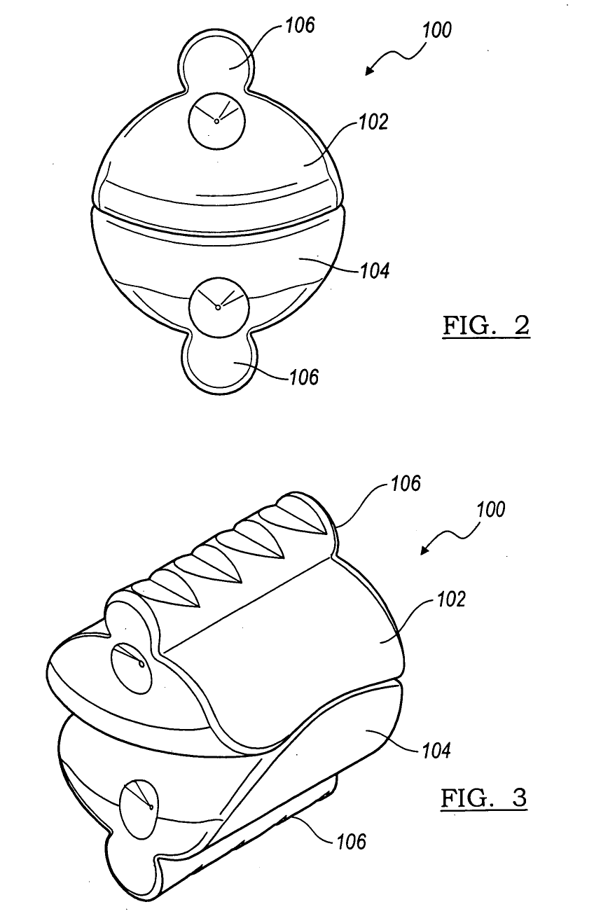

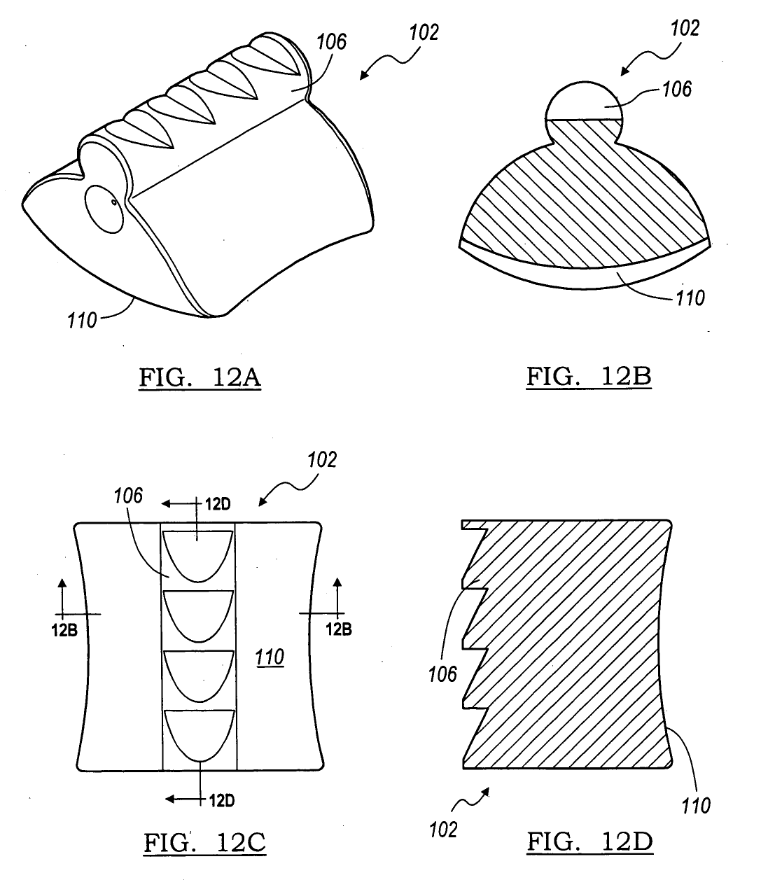

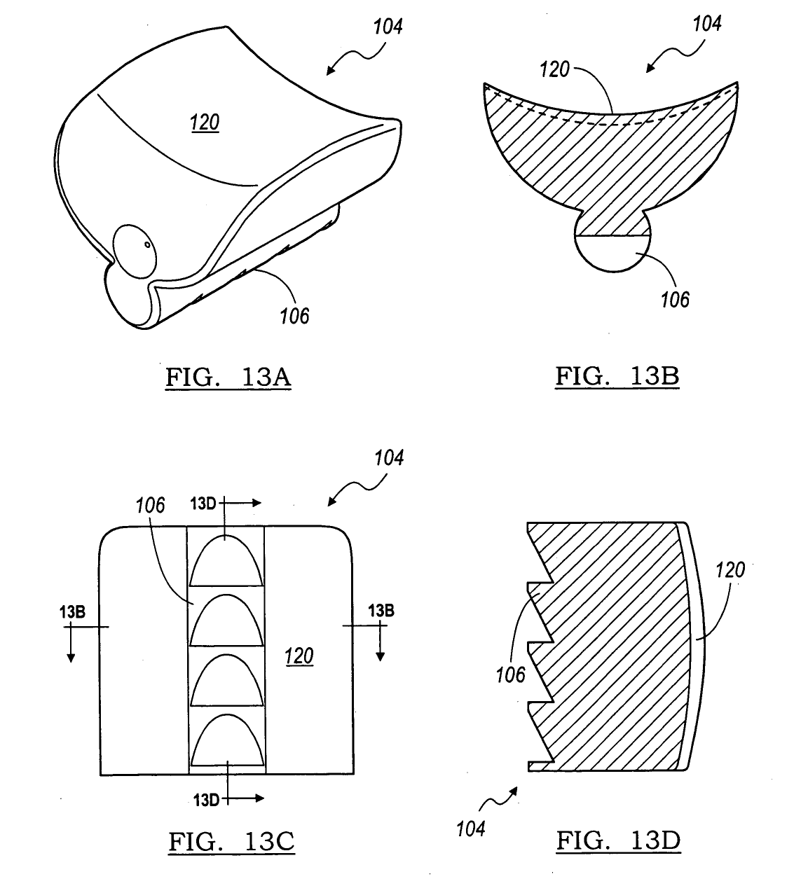

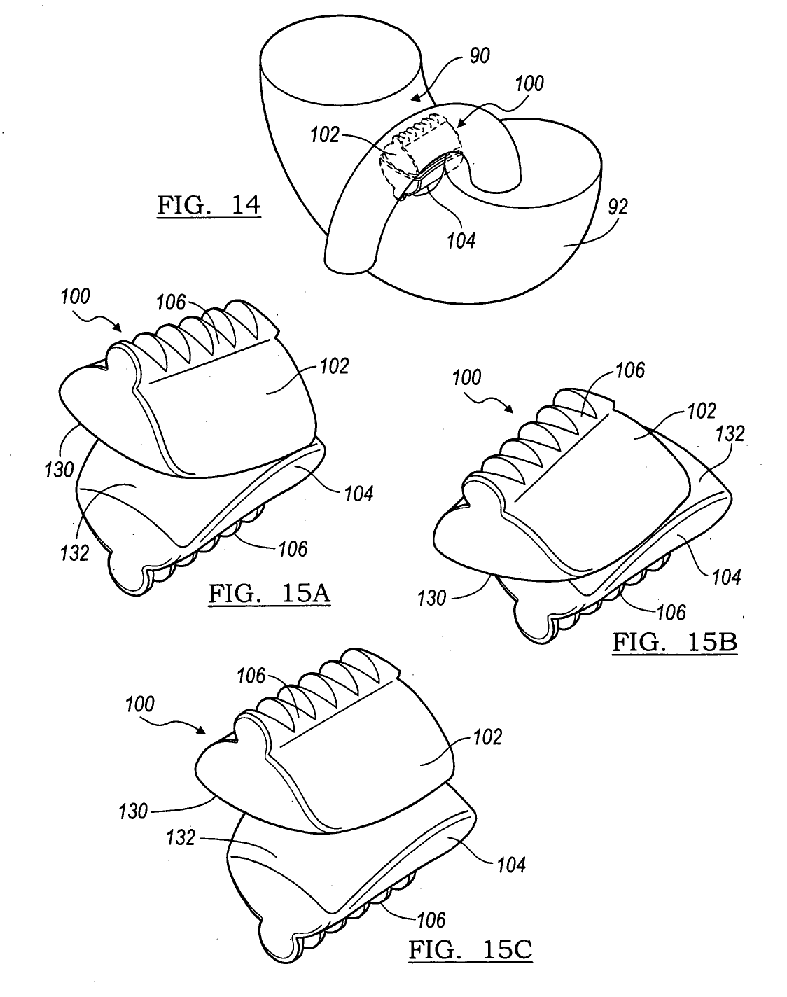

Referring to FIGS. 2, 3, and 12-14, the toroidal intervertebral implant 100 can be created, for example, by removing a cylinder at the contact between two torii 90, 92, as conceptually illustrated in FIG. 14. Referring to FIG. 12C, the superior component 102 includes an articulating surface 110. The articulating surface 110 of the superior component 102 includes a convex radius in the coronal plane, as shown in FIG. 12B, and a concave radius in the sagittal plane, as shown in FIG. 12D. Referring to FIG. 13C, the inferior component 104 includes an articulating surface 120. The articulating surface 120 of the inferior component 104 includes a concave radius in the coronal plane, shown in FIG. 13B, and a convex radius in the sagittal plane, shown in FIG. 13D. In the sagittal plane, the superior articulating surface 110 can have a larger radius of curvature than the inferior articulating surface 120. In one aspect, in the coronal plane, the convex superior articulating surface 110 can be defined by a shallow “V” having a tip that is rounded with a fillet radius. The toroidal intervertebral implant 100 can include an A/P taper to minimize subchondral bone removal.

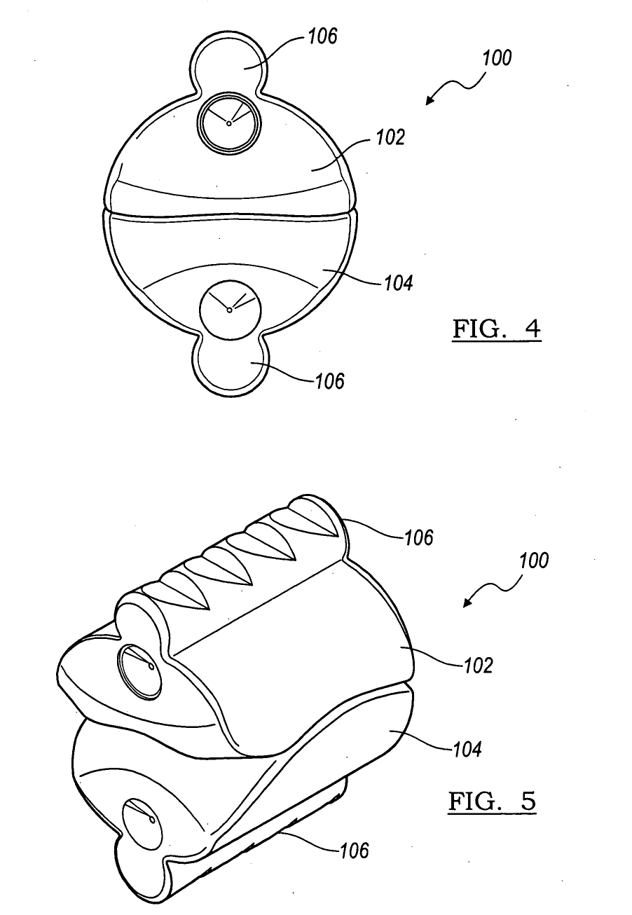

Referring to FIGS. 4, 5, and 16, the superior and inferior components 102, 104 of the spherical intervertebral implant 100 include respective articulating surfaces 130, 132. The articulating surface 132 of the inferior component 104 is convex and at least partially spherical. The articulating surface 130 of the superior component 102 is concave. In the sagittal plane, shown in FIG. 16A, the radius of the superior component 102 can be greater than the radius of the inferior component 104 to allow for anterior-posterior (A/P) translation. The apex of the articulating surfaces 130, 132 is indicated by axis A-A in FIG. 16A, and can be offset two thirds posteriorly to align the articulating fulcrum of the spherical intervertebral implant 100 with the nuclear recess in the vertebral endplates 84. The radius of curvature of the inferior articulating surface 132 can be larger anteriorly to the apex (axis A-A) than the radius of curvature posteriorly to the apex, as illustrated in FIG. 16A. The spherical intervertebral implant 100 can include an A/P taper to minimize subchondral bone removal. In the coronal plane, shown in FIG. 16B, the curvatures of the articulating surfaces 130, 132 are congruent with equal radii to maximize contact area.

The intervertebral implant 100 illustrated in FIGS. 1A, and 17A-17C, can have a spherical or toroidal type of articulation, as discussed above, although spherical articulating surfaces 301, 303 are illustrated. The superior and inferior articulating components 102, 104 can include respective superior and inferior bone engagement surfaces 305, 309. The superior and inferior bone engagement surfaces 305, 309 can include pairs of separate outwardly convex end portions 306, 308 connected with outwardly concave intermediate portions 304, 310, respectively. The superior and inferior bone engagement surfaces 305, 309 can be formed, for example, by two cylinders 306a, 306b of circular cross-section, which can be intersecting, as illustrated in FIG. 17B in dotted lines. Accordingly, the outer surface 101 of the bi-cylindrical intervertebral implant can be defined as a curved surface enveloping the intersecting cylinders 306a, 306b. Non-intersecting cylinders can also be used in other aspects.

Each of superior and inferior bone engagement surfaces 305, 309 can include bone-engagement formations 302. The bone engagement formations 302 can arranged in parallel rows on the convex end portions 306, 308. The engagement formations 302 can include crests 312 and grooves 314. Both crests 312 and grooves 314 can be designed with smooth rounded profiles balancing effective bone engagement while reducing potential damage by avoiding sharp edges.

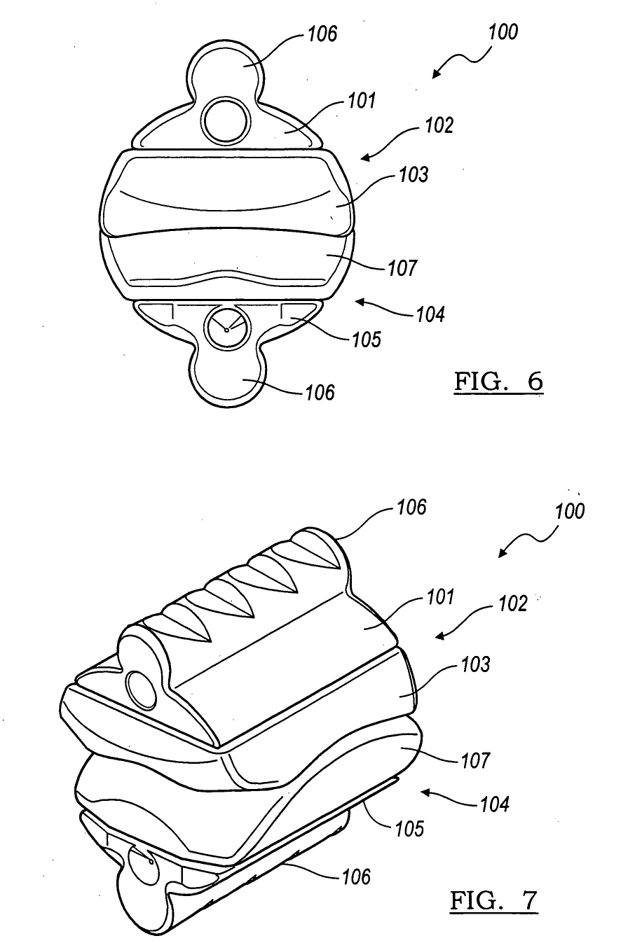

The intervertebral implant 100 can be manufactured from biocompatible materials, such as, for example, cobalt chromium alloy, titanium alloys or other metals, pyrolytic carbon, and other materials. It can also be constructed from a combination of materials. Referring to FIGS. 6 and 7, each superior component 102 can include an outer portion 101 made of titanium, titanium alloy or other biocompatible metal or alloy, and an articulating portion 103 made of pyrolytic carbon. Similarly, each inferior component 104 can include an outer portion 105 made of titanium, titanium alloy or other biocompatible metal or alloy, and an articulating portion 107 made of pyrolytic carbon. It should be noted that although the intervertebral implant 100 illustrated in FIGS. 6 and 7 is of the spherical type, the toroidal intervertebral implant 100 can also be manufactured by a similar combination of materials. It will be appreciated that other biocompatible metallic or non-metallic materials can also be used.

It will be appreciated that the terms “toroidal” and “spherical” are in reference to the relative articulation of the superior and inferior components 102, 104, and that the overall shape of the intervertebral implant 100 can substantially cylindrical, as illustrated 2, 3 and 6, or bi-cylindrical, as illustrated in FIGS. 17A-17C. Referring to FIGS. 2, 3, and 6, the coronal section of the intervertebral implant 100 can include a substantially circular central section defined by the superior and inferior components 102, 104 and two partially circular extensions defined by the serrated racks 106. It will be appreciated, however, that particular features associated with particular illustrations are merely exemplary. According features that illustrated in one exemplary embodiment can also be used in other embodiments, although not particularly illustrated.

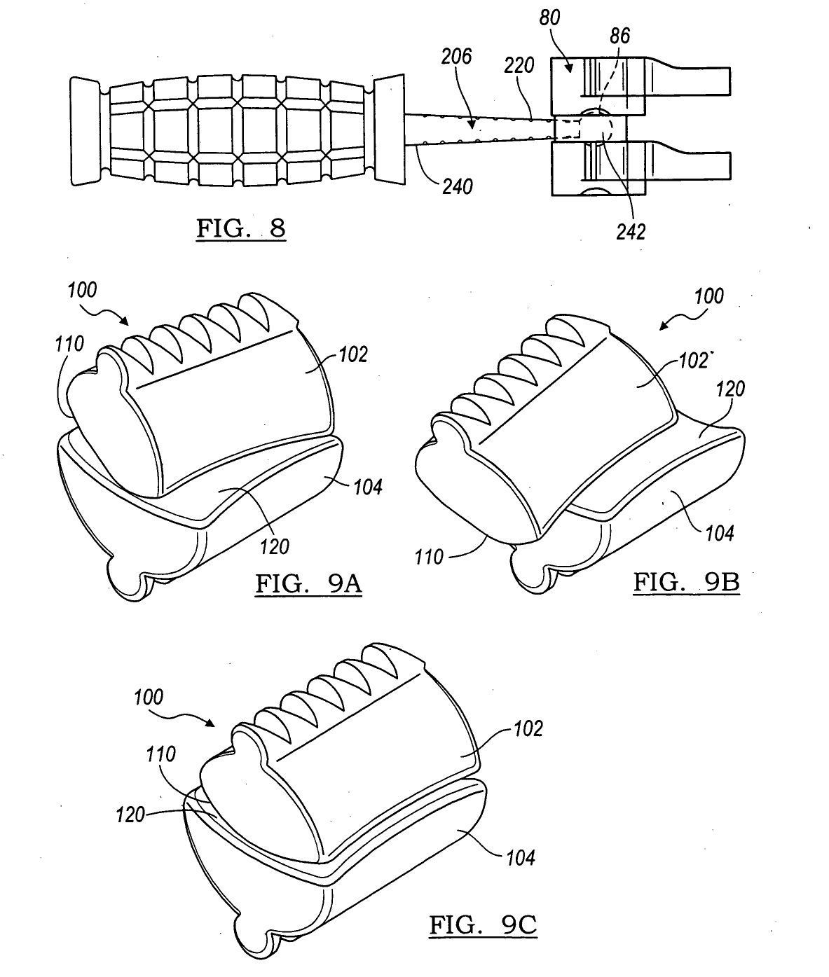

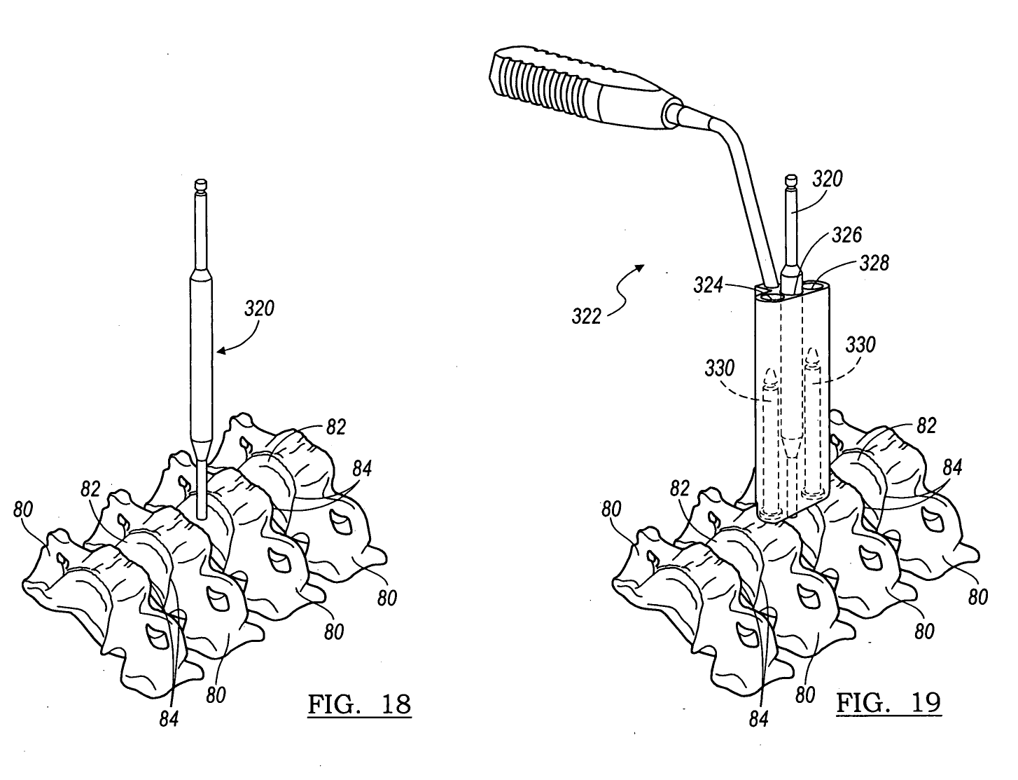

The method of implanting the intervertebral implant 100 and associated instruments is described with particular reference to FIGS. 18-25, and with additional reference to FIG. 8, for implanting the intervertebral implant 100 illustrated in FIGS. 17A-17C.

Preparatory to the surgical procedure, the patient can be positioned such that there is a natural amount of lordosis, if the surgeon prefers to perform a discectomy under distraction. The affected segment of the spine can be exposed anteriorly. A small annulotomy/discectomy can be performed, excising the nucleus and all degenerated material. Referring to FIG. 18, the annulotomy/discectomy can be sized for receiving a centering shaft 320 or other centering/locating instrument, such as, for example, a fossa locator 206 illustrated in FIG. 8. The fossa locator 206 can be inserted into the natural disc space to locate the nuclear recess 86. The fossa locator 206 can include a removable handle 208 include a shaft 240 and a distal tip 242 that can be cylindrical in shape. The fossa locator 206 can be inserted until the tip 242 engages the nuclear recess 86. Graduated markings 220 on the shaft 240 of the fossa locator 206 indicate the depth required for subsequent drilling and broaching. The handle 208 from the fossa locator 206 can then be removed, such that the shaft 240 of the fossa locator can also function as a centering shaft, such as the centering shaft 320 illustrated in FIG. 18.

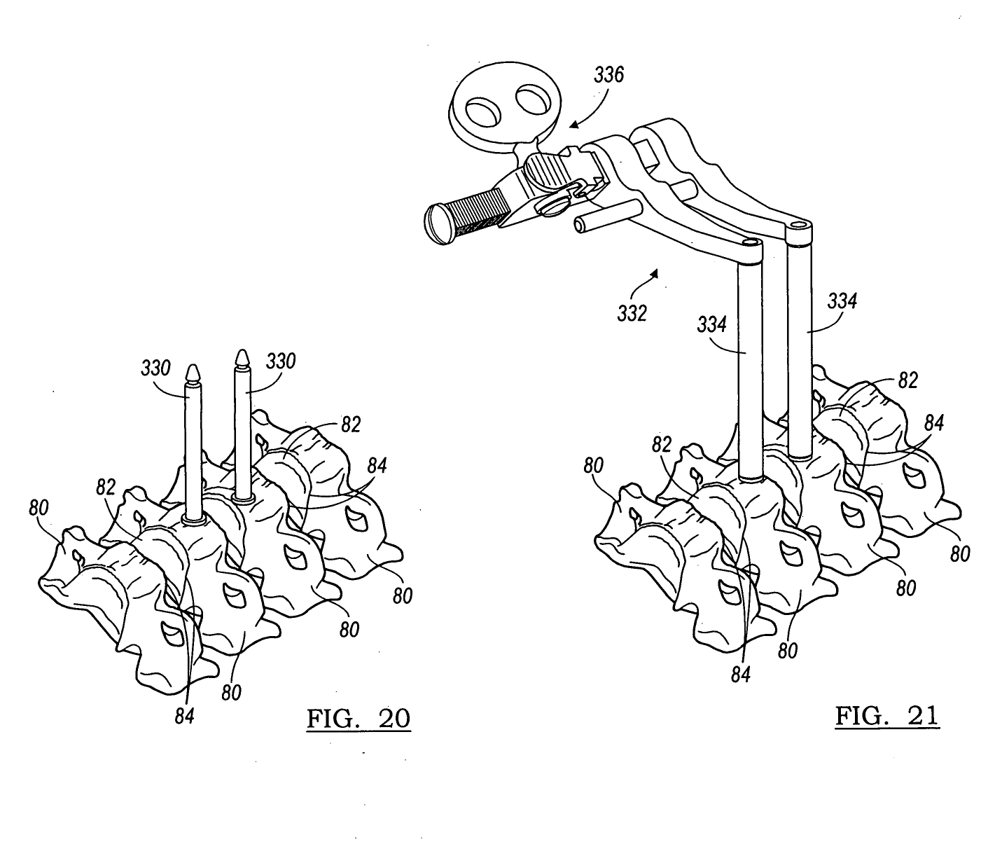

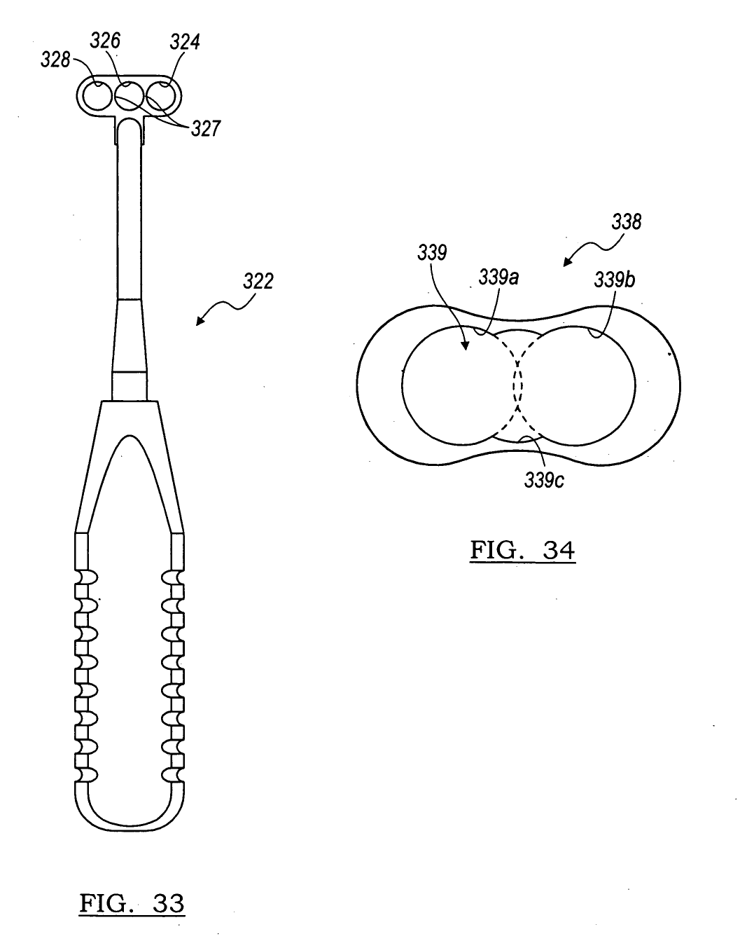

Referring to FIGS. 19 and 33, a distraction pin guide 322 can be placed over the centering shaft 320. The distraction pin guide 322 can include a pair of side longitudinal openings/lumens 324, 328 and an intermediate longitudinal opening/lumen 326 positioned therebetween. The intermediate longitudinal opening 326 can be defined by an internal wall structure 327 that fully separates the intermediate opening 326 from the side openings 324, 328, as illustrated in FIG. 33, which shows the intermediate opening 326 and the side openings 324, 328 as three non-intersecting circles. It will be appreciated that other wall structures can also be used, including wall structures that allow at least partial communication between the intermediate opening 326 and the side openings 324, 328. The centering shaft 320 can be received in the intermediate opening 326, which is appropriately sized. A pair of self-drilling distraction pins or other anchoring pins 330 can be inserted through the side openings 322, 328 for anchoring into adjacent vertebrae on opposite sides of the disc space. The centering shaft 320 and the distraction pin guide 322 can be removed after placement of the distraction pins 330, as illustrated in FIG. 20.

Referring to FIGS. 21-30, a distractor 332 can be used for facilitating the implantation procedure. The distractor 332 can include a pair of tubular legs 334 and a distraction mechanism 336 for applying and controlling the amount of distraction, if any, desired by the surgeon. The distractor legs 334 can be placed over the pins 330, as illustrated in FIG. 21. The depth of inferior vertebral body can be measured using a depth gauge, such as the fossa locator 206 illustrated in of FIG. 8. This measurement can be used to determine the drilling depth.

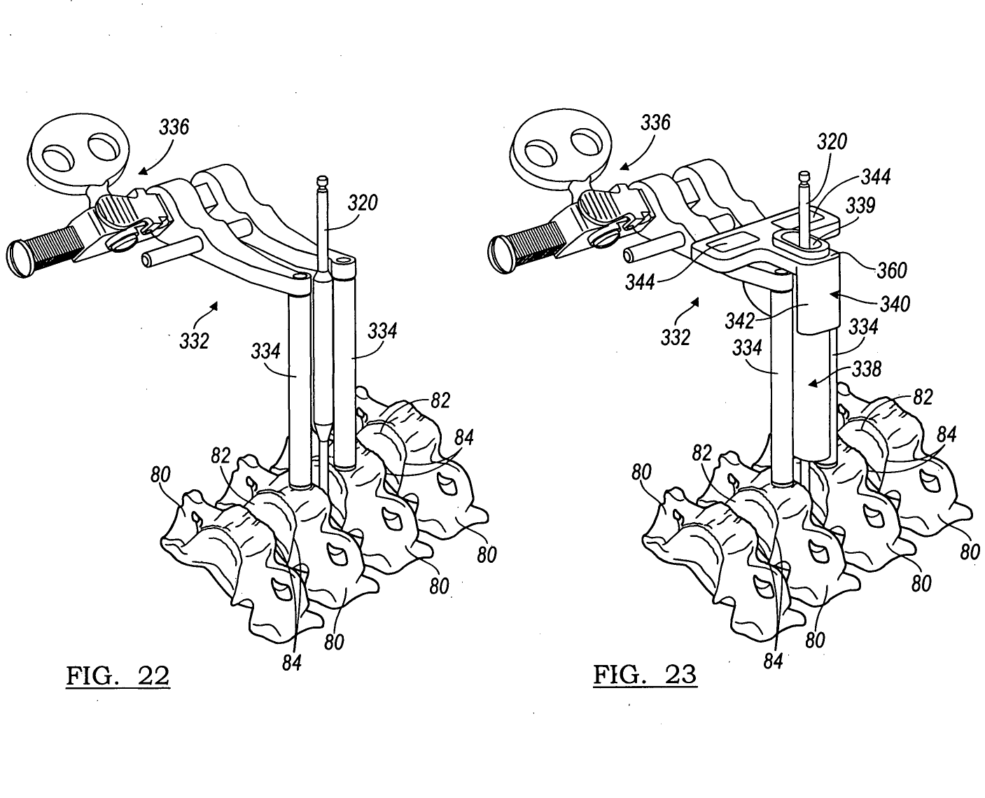

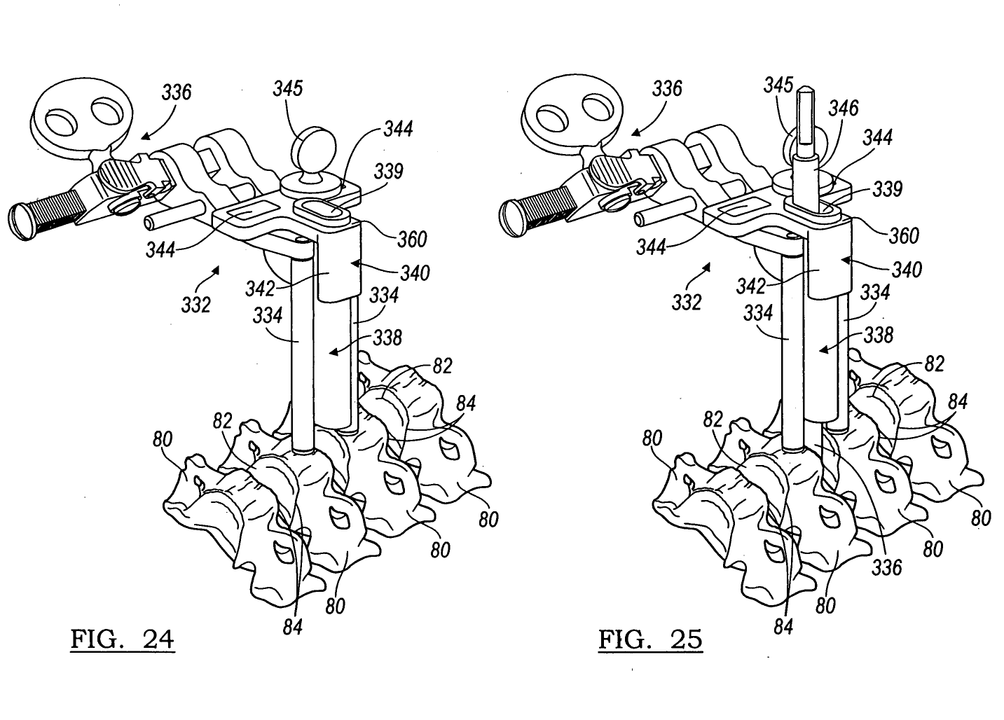

Referring to FIGS. 22-27 and 34, the centering shaft 320 can be inserted into the disc space. A drill guide cannula 338 can be positioned over the centering shaft 320 and between the legs 334 of the distractor 332. The drill guide cannula 338 can be secured on the distractor 332 with a cannula lock 340. The cannula lock 340 can include a longitudinal element 342 defining a first opening 361 configured for receiving the drill guide cannula 338 therethrough, and a flange 360 at an angle to the longitudinal element 342. The flange 360 can define one or more flange openings 344 for engaging a locking element 345, such as a thumb screw. The drill cannula 338 can be pre-assembled in the cannula lock 340 through the first opening 361, and the assembly can be placed over the centering shaft 320. The flange 360 of the cannula lock 340 can sit on the distractor 332, and the drill guide cannula 338 can be secured on the distractor 332 by tightening the locking element 345 through one of the flange openings 344. The drilling depth can be measured by reading markings provided on the centering shaft 320 at the top of the drill guide cannula 338, as described above in connection with the fossa locator 240 illustrated in FIG. 8, and compared with the required drilling depth determined earlier. After the drilling depth is confirmed, the centering shaft 320 can be removed, as shown in FIG. 24.

Referring to FIGS. 25-27 and 34, the drill guide cannula 338 can include a longitudinal opening 339 adapted for receiving the centering shaft 320 for locating guidance, and other instruments, such as a drill 346 which can be inserted in more than one position relative to the longitudinal opening 339, as appropriate for preparing the disc space for accommodating the overall geometry of the particular intervertebral implant 100. For example, for the bi-cylindrical intervertebral implant 100 illustrated in FIGS. 1A, and 17A-17C, the drill 346 can be positioned in first and second positions defined by first and second open intersecting circles 339a, 339b of the longitudinal opening 339 of the drill guide cannula 338, as illustrated in FIG. 34, and corresponding to the circles 309a, 309b of the bi-cylindrical intervertebral implant 100 illustrated in FIG. 17B. The centering shaft 320 can be received in an intermediate position defined by a third circle 339c of smaller diameter than the first and second circles 339a, 339b, and intersecting the first and second circles 339a, 339b, as illustrated in FIG. 34.

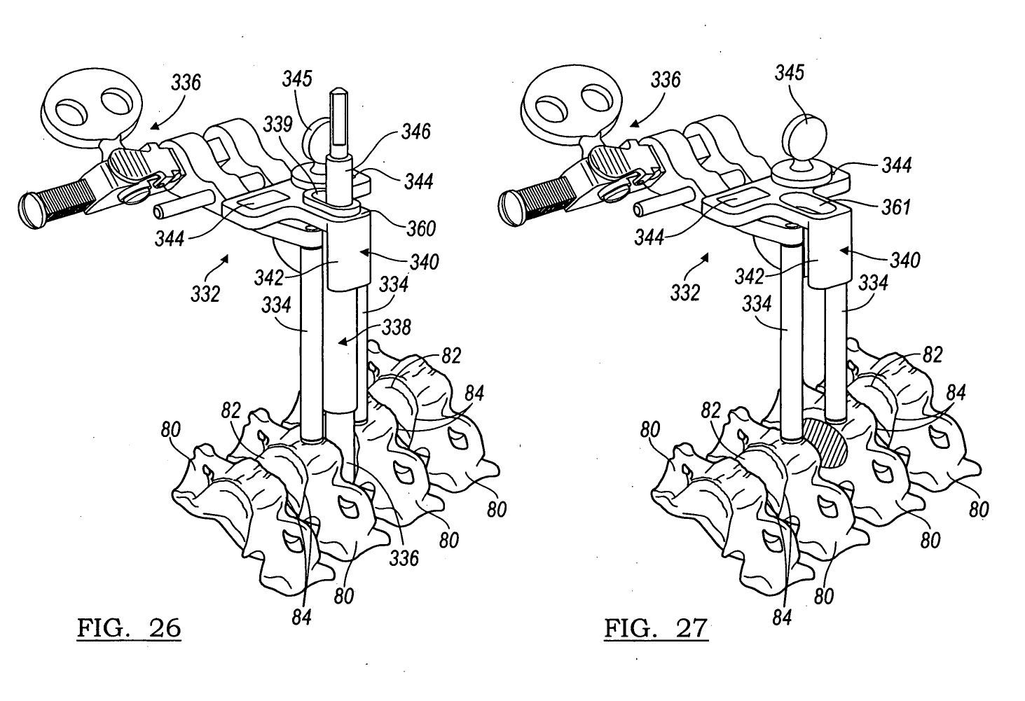

In one exemplary embodiment, flat-bottomed holes having diameter of about 8 mm can be drilled to a depth determined as described above. Drill stops can be used to control the depth of drilling and/or broaching. The desired depth can align the center of the intervertebral implant 100 with the nuclear recess 86. After drilling, bone debris can be removed by irrigation and suction, and the drill guide cannula 338 can be pulled out of cannula lock 340 and completely removed, as illustrated in FIG. 27. The drill guide cannula 338 can be sized such that it stops short of the vertebrae defining a gap 362 between the distal end of the cannula 338 and the vertebrae, as can be seen in FIG. 26. The gap 362 can facilitate the removal of the drill guide cannula 338 after drilling.

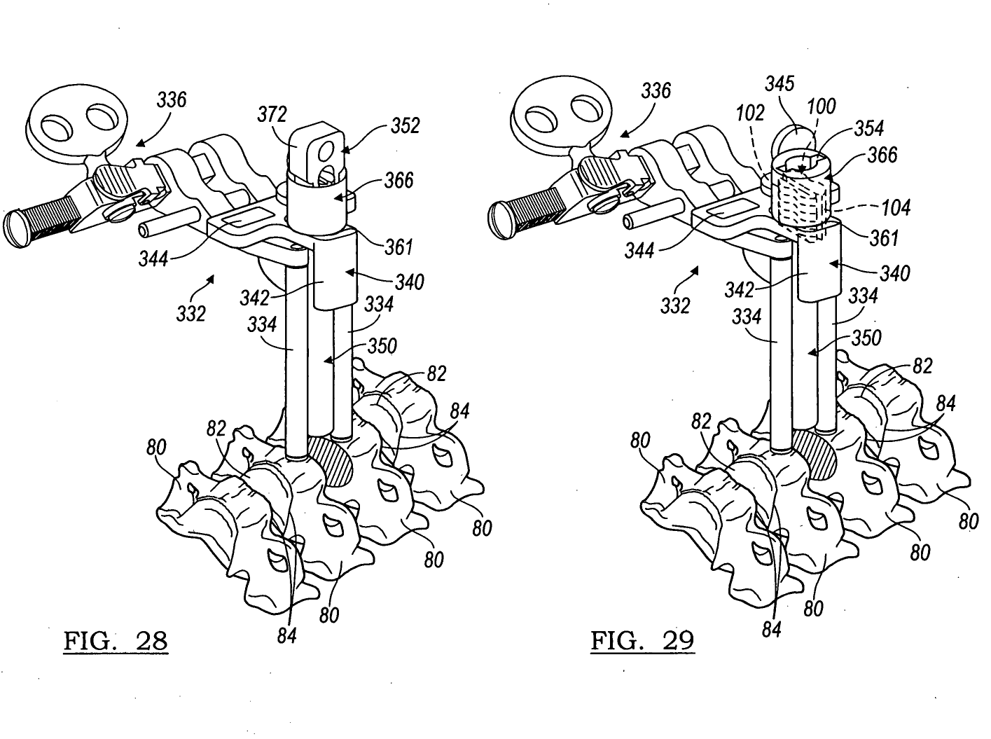

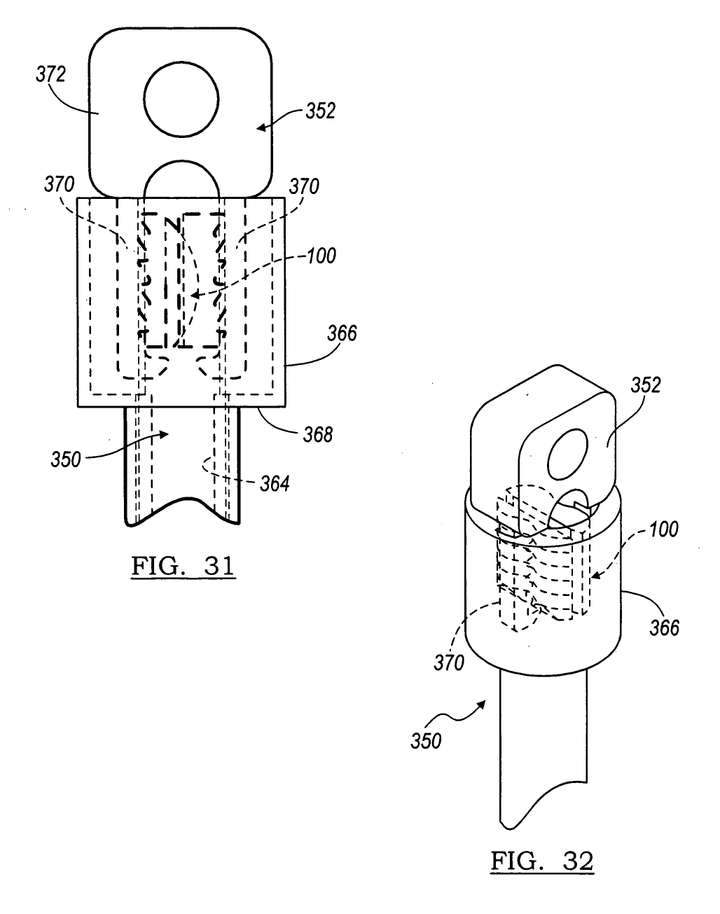

Referring to FIGS. 28-32, an elongated insertion cannula 350 can be inserted into the first opening 361 of the cannula lock 340. The insertion cannula 350 can be pre-loaded with the intervertebral implant 100, as illustrated in FIGS. 31 and 32. The insertion cannula 350 can be made of smooth plastic that can protect the intervertebral implant 100 from scratching, for example, and can be disposable. The insertion cannula 350 can include a longitudinal bore 364. The longitudinal bore 364 can be shaped to conform to, and/or otherwise accommodate the shape of intervertebral implant 100, for example the bi-cylindrical implant the intervertebral implant 100, as illustrated in FIG. 31. The shape of the longitudinal bore 364 can also maintain the relative position of the components 102, 104 of the multiple-component intervertebral implant 100. The insertion cannula 350 can include an enlarged tubular proximal end 366, which can provide a shoulder 368 resting on the cannula lock 340 when the insertion cannula 350 is inserted through the first opening 361 of the cannula lock 340.

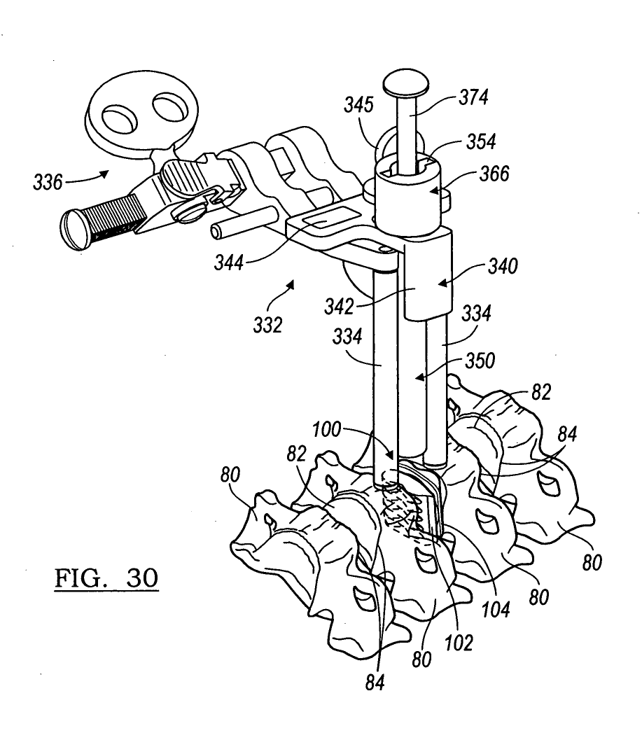

Referring to FIGS. 28-32, the intervertebral implant 100 can be held in the enlarged proximal end 366 of the insertion cannula 350 using a removable retainer or other temporarily retaining device, such as a clip 352, for example. The clip 352 can hold the intervertebral implant 100 at a substantially fixed position within the longitudinal bore 364 of the insertion cannula 350, and maintain the relative positions of the superior and inferior components 102, 104 of the intervertebral implant 100. The clip 352 can be substantially flat and can include a head 372 and two compliant arms 370 extending from the head 372. The compliant arms 370 that can hold the intervertebral implant 100 at the concave intermediate portions 304, 310 of the intervertebral implant 100 shown in FIG. 17B. The arms 370 can be received through a diametrical slot 354 of the proximal end 366 of the insertion cannula 350, or other appropriate opening thereon. The clip 352 can be inserted from the proximal end 366 of the insertion cannula 350, and can be removed by pulling out the proximal end 366. Removing the clip 352 causes the arms 370 to open, thereby releasing the intervertebral implant 100 into the bore 364 of the insertion cannula 350. A plastic tamp 374 can be used to push the intervertebral implant 100 through the insertion cannula 350 and into the prepared disc space, as illustrated in FIG. 30. The insertion cannula 350, the distractor 332 and the distraction pins 330 can then be removed leaving the intervertebral implant 100 appropriately positioned, as illustrated in FIG. 1A.

The intervertebral implant 100 can be provided in a sterilized kit that includes the insertion cannula 350. The intervertebral implant 100 can be preloaded in the insertion cannula 350 and held by the clip 352. The tamp 374 can also be included in the kit. Kits including intervertebral implants 100 of different sizes can be provided. After use, any of the insertion cannula 350, the clip 352 and the tamp 374 can be disposed, or re-sterilized and re-used.

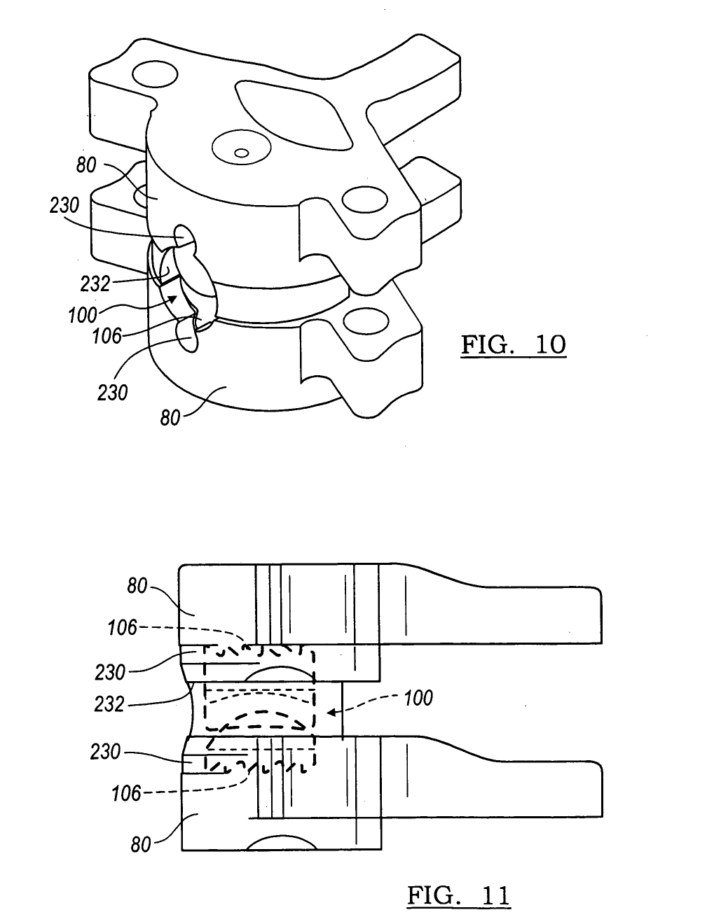

Although the method of implanting the intervertebral implant 100 and associated instruments was described above in reference to the bi-cylindrical intervertebral implant 100 illustrated in FIGS. 17A-17C, similar procedures can be used for implanting the toroidal and spherical intervertebral implants 100 illustrated in FIGS. 2, 4 and 6. Referring to FIGS. 10 and 11, for example, a pair of holes 230 can be drilled to the required depth as determined by the graduated markings 220 of the fossa locator 206 for accommodating the serrated racks 106 of the toroidal or spherical intervertebral implant 100. A central hole 232 can be drilled per the required depth to accommodate the body of toroidal or spherical intervertebral implant 100. Similarly, the shape of the various implantation instruments, such as the drill guide cannula and the insertion cannula, for example, can be designed to accommodate the toroidal or spherical implant.

The method of implanting the intervertebral implant 100 can be used, at the option of the surgeon, for minimally invasive procedures, using a small incision and removing only as much degenerative material as necessary. Accordingly, a decreased risk of infection, decreased blood loss, decreased exposure to anesthesia and shorter recovery time can be achieved.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention.

What is claimed is:

1. An intervertebral implant comprising:
a first component having a first articulating surface and a first bone engagement surface for engaging a first vertebra; and
a second component having a second articulating surface and a second bone engagement surface for engaging a second vertebra adjacent to the first vertebra, wherein the second articulating surface articulates with the first articulating surface for substantially replicating a natural spinal movement including torsion, extension/flexion, and lateral bending, and wherein the first and second bone engagement surfaces define an outer surface substantially shaped as an envelope of two intersecting cylinders.

2. The intervertebral implant of claim 1, wherein each of the first and second bone engagement surfaces comprises a pair of separate convex end portions connected with a concave intermediate portion.

3. The intervertebral implant of claim 2, wherein the first and second articulating surfaces have substantially equal radii of curvature in a coronal plane and different radii of curvature in a sagittal plane.

4. The intervertebral implant of claim 3, wherein the first articulating surface includes a concave portion in the coronal and sagittal plane, and the second articulating surface includes a convex portion in the coronal and sagittal plane.

5. The intervertebral implant of claim 2, wherein the first articulating surface comprises a convex portion in the coronal plane and a concave portion in the sagittal plane, and the second articulating surface includes a concave portion in the coronal plane and convex portion in the sagittal plane.

6. The intervertebral implant of claim 5, wherein in the sagittal plane the curvatures of the respective convex and concave portions of the first and second articulating surfaces are different.

7. The intervertebral implant of claim 5, wherein in the coronal plane the first articulating surface is substantially V-shaped with a rounded tip.

8. The intervertebral implant of claim 2, further comprising bone engagement formations arranged in substantially parallel rows on the first and second bone engagement surfaces.

9. The intervertebral implant of claim 1 in combination with an insertion cannula preloaded with the intervertebral implant.

10. A surgical kit comprising:
an insertion cannula defining a longitudinal bore;
an intervertebral implant pre-loaded within the longitudinal bore; and
a retainer for temporarily retaining the intervertebral implant within the longitudinal bore.

11. The surgical kit of claim 10, wherein the retainer comprises a clip having at least one compliant arm for retaining the intervertebral implant in the insertion cannula.

12. The surgical kit of claim 11, wherein the insertion cannula comprises a proximal end portion configured for coupling with the clip.

13. The surgical kit of claim 10, wherein the insertion cannula is constructed of plastic.

14. The surgical kit of claim 10, wherein the spinal implant is a multiple component implant and the bore is shaped to maintain the relative positions of the multiple components during implantation.

15. A method for inserting an intervertebral implant in a disc space, the method comprising:
providing an insertion cannula having a longitudinal bore;
preloading the intervertebral implant within the longitudinal bore of the insertion cannula in a substantially fixed position;
supporting the insertion cannula relative to the disc space;
releasing the intervertebral implant from the substantially fixed position; and
implanting the intervertebral implant into the disc space.

16. The method of claim 15, wherein preloading the intervertebral implant comprises holding the intervertebral implant with a compliant clip coupled to a proximal end of the insertion cannula.

17. The method of claim 16, wherein releasing the intervertebral implant comprises removing the compliant clip.

18. The method of claim 15, wherein supporting the insertion cannula comprises supporting the insertion cannula on a distractor coupled to vertebrae adjacent the disc space.

19. The method of claim 15, further comprising locating a nuclear recess of the disc space.

20. The method of claim 20, wherein the implant is a multiple component implant and wherein preloading the implant comprises maintaining the relative positions of the multiple component implant.
